(19) United States
(12) Patent Application Publication
(10) Pub. No.: US 2018/0280111 A1
(43) Pub. Date: Oct. 4, 2018

(54) Biopsy Site Marker

(71) Applicant: David Parish, Southlake, TX (US)

(72) Inventor: David Parish, Southlake, TX (US)

(21) Appl. No.: 15/938,257

(22) Filed: Mar. 28, 2018

Related U.S. Application Data
(60) Provisional application No. 62/479,779, filed on Mar. 31, 2017.

(51) Int. Cl. A61B 90/00; A61B 8/08

(52) U.S. Cl. CPC A61B 90/39; A61B 8/488; A61B 2090/3925; A61B 8/0833; A61B 2090/3908

(57) ABSTRACT

The present disclosure relates to apparatus and methods for improving visualization of biopsy site markers under ultrasound. In some implementations, the method for detecting a biopsy site marker in tissue may comprise inserting a biopsy site marker having a textured and/or irregular surface into the tissue, imaging the biopsy site marker using Doppler sonography, and identifying the marker based on twinkling artifact appearing on the Doppler sonography. In other implementations, the apparatus of the present disclosure may comprise a biopsy site marker for placement into human tissue, the marker having at least one textured or irregular surface capable of creating twinkling artifact when imaged using Doppler sonography.

### CROSS-REFERENCE TO RELATED APPLICATIONS

This application claims the benefit of, and priority to, U.S. Provisional Patent Application Ser. No. 62/479,779, filed Mar. 31, 2017 and entitled “Biopsy Site Marker”, the content of which is incorporated herein by reference for all purposes.

### BACKGROUND

A biopsy is a medical procedure in which one or more small tissue samples are extracted from the body for purposes of testing. After extraction of the tissue samples, a biopsy site marker may be deposited at the biopsy site to “mark” the area for subsequent identification. Biopsy site markers, which are generally small, echogenic structures, are often poorly visualized under ultrasound because they are not easily distinguished from other echogenic structures in the body such as fibrous tissue, fatty tissue, ducts, and the like.

### SUMMARY

The present disclosure relates to apparatus and methods for improving the visibility of biopsy site markers during ultrasound and other similar medical imaging.

In some implementations, the method for detecting a biopsy site marker in human tissue may comprise inserting a biopsy site marker having a textured and/or irregular surface into the tissue, imaging the biopsy site marker using Doppler sonography, and identifying the marker based on twinkling artifact appearing on the Doppler sonography.

In other implementations, the apparatus of the present disclosure may comprise a biopsy site marker for placement into human tissue, the marker having at least one textured or irregular surface capable of creating twinkling artifact when imaged using Doppler sonography.

The details of one or more implementations are set forth in the accompanying drawings and the description below. Other features, objects, and advantages of the implementations will be apparent from the description and drawings.

### BRIEF DESCRIPTION OF THE DRAWINGS

For a more complete understanding of this disclosure and its features, reference is now made to the following description, taken in conjunction with the accompanying drawings, in which:

FIG. 1 illustrates an implementation of a biopsy site marker in accordance with the present disclosure;

FIG. 2 illustrates another implementation of a biopsy site marker in accordance with the present disclosure;

FIG. 3 illustrates another implementation of a biopsy site marker in accordance with the present disclosure;

FIG. 4 illustrates another implementation of a biopsy site marker in accordance with the present disclosure;

FIG. 5 illustrates another implementation of a biopsy site marker in accordance with the present disclosure.

### DETAILED DESCRIPTION

The present disclosure is generally directed to apparatus and methods relating to detecting a biopsy site marker in human and/or other mammalian tissue. The biopsy site marker and method of detecting thereof may comprise a plurality of different features and components as described herein.

A biopsy is a common medical procedure in which small tissue samples, known as biopsy specimens, are removed from organs, muscles, suspected tumors, lesions, or other tissues of the body. The removed specimens are then subjected to diagnostic testing to determine cytology, histology, malignancy, or the presence or absence of substances that act as indicators for disease. Core needle biopsy is a standard image-guided procedure performed using mammography, MRI, or ultrasound imaging, wherein a hollow needle is used to withdraw small cylinders (or cores) of suspicious or potentially abnormal tissue from the body for testing and analysis. After extraction of the tissue sample(s), a marker is deposited at the biopsy site to “mark” the area. Made of titanium or stainless steel, biopsy site markers are typically 3-4 millimeters in size. After a core needle biopsy is performed, post-procedure imaging confirms that the biopsy site marker has been placed in the appropriate location in the tissue. Thus, one of the primary advantages of biopsy site markers is to enable medical practitioners to identify the biopsied area at a later time, i.e., if the abnormal tissue needs to be re-evaluated or surgically removed weeks, months, or even years later.

If the biopsied area is required to be removed surgically, a procedure called wire localization may be performed. This procedure is image-guided and requires the insertion of a metal wire next to the existing biopsy site marker. First, the biopsy site marker must be identified by, for example, ultrasound, MRI, or mammographic imaging. Then, the distal tip of the metal wire is inserted into the tissue and placed adjacent to the marker. The proximal portion of the wire comes out of the tissue through the skin. Additional images (via ultrasound, MRI, or mammogram) are taken to confirm that the wire is correctly positioned adjacent to the marker. The patient is then sent to surgery. Used as a guidance tool by the surgeon to access the problematic tissue, the wire is removed along with the surrounding tissue.

While wire localizations may be performed by ultrasound, mammography or MRI, ultrasonography is advantageous for many reasons. First, in contrast to mammograms, ultrasound imaging does not use radiation and does not require uncomfortable positioning or compression of breast tissue. Moreover, unlike MRIs, ultrasound imaging does not require uncomfortable prone positioning and is less expensive. As such, ultrasound imaging may often be preferred over other imaging modalities.

However, while metallic biopsy site markers may be visible on Mill or mammographic x-ray imaging, they are poorly visualized under ultrasound. This is because, under ultrasound, the small echogenic metallic markers are not easily distinguished from other echogenic structures in the body such as fibrous tissue, fatty tissue, ducts in breast tissue, and the like.

Traditional methods to increase the visibility of biopsy site markers under ultrasound include embedding the metal site markers within large absorbable materials. For example, in certain applications, the biopsy site marker may be surrounded by mesh, sponge, or gel material which expand when in contact with tissue fluid, thereby allowing the biopsy site marker to be more readily seen under ultrasound. However, one of the disadvantages of these applications is that the absorbable material covering the marker is absorbed by the body within 12-15 months, leaving the metal site marker once again undetectable under ultrasound. Another disadvantage of the prior art applications is the bulkiness of the absorbable material. If inserted close to the surface of the skin, the bulky site marker may be externally palpable and uncomfortable for the patient.

The apparatus and methods of the present disclosure accomplishes permanency and visibility while also avoiding bulkiness of biopsy site markers by utilizing “Doppler aliasing artifact” (hereinafter referred to as “twinkling artifact”)—a color phenomenon visible on Doppler ultrasound. Color Doppler mode in clinical diagnostic ultrasound is traditionally used to detect motion, particularly blood flow. Different colors are assigned to different blood flow speeds. Red is most often used for high arterial blood flow and blue is used for slower venous blood flow. However, although Color Doppler was designed to view and measure flow patterns, it has been documented that when certain stationary masses in the body—such as kidney stones or calcifications—are imaged in Doppler mode, the masses are displayed as a rainbow of rapidly alternating colors, i.e., twinkling artifact, thereby enabling immediate detection.

While the source and mechanism of the artifact is not definitively known, several published studies have offered hypotheses. For example, in 1996, Rahmouni et al. (“Color Doppler Twinkling Artifact in Hyperechoic Regions”) proposed that twinkling artifact is generated by rough surfaces with multiple reflectors which split the incident beam generated by the ultrasound machine into a complex beam pattern, creating an increased pulse duration of the received radio frequency signal which then shows as movement. By contrast, in 2002, Kumaya et al. (“Twinkling Artifact on Color Doppler Sonography: Dependence on Machine Parameters and Underlying Cause”) offered that the appearance of the twinkling artifact is instead highly machine- and setting-dependent, i.e., that the narrow bandwidth noise introduced by phase jitter in Doppler machine circuitry impacts artifact appearance and that surface roughness magnifies the artifact. In a more recent 2013 study, Lu et al. (“Evidence for trapped surface bubbles as the cause for twinkling artifact in ultrasound imaging”) opined that twinkling artifact is caused, not by an abnormal response of machine electronic circuits, but by small bubbles that are trapped and stabilized in cracks and crevices on a rough reflective surface. These publications show that although the precise cause of twinkling artifact is not known, it is evident that the phenomenon is a useful tool in locating kidney stones and other biological masses which are otherwise difficult to visualize under ultrasound.

The present disclosure purposes to use the twinkling artifact to improve the visibility of biopsy site markers during ultrasound and other similar medical imaging.

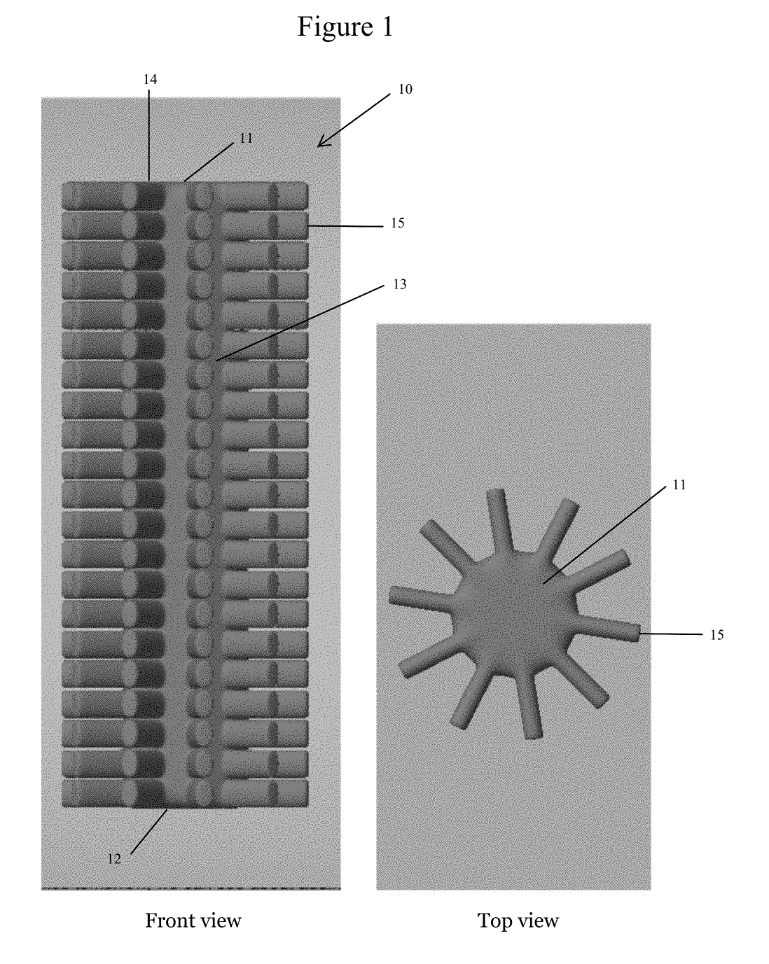

FIG. 1 is a biopsy site marker 10 according to an implementation of the present disclosure. Biopsy site marker 10 may comprise a metallic, rod-shaped cylinder having a top surface 11, a bottom surface 12, and a body 13. Body 13 is approximately three to four millimeters in length. The top and bottom surfaces of biopsy site marker 10 are each approximately 1.1 mm in diameter. Extending radially from the body 13 are vertical rows 14 of spokes. Each row 14 comprises an equally numbered plurality of spokes 15 positioned adjacent to and abutting one another. Each vertical row 14 is equilaterally and radially spaced around the body 13 of biopsy site marker 10. The plurality of spokes 15 of biopsy site marker 10 results in a textured and reflective surface morphology that may reliably reproduce twinkling artifact when imaged under Doppler ultrasound, thereby improving visibility of the biopsy site marker 10 on ultrasound.

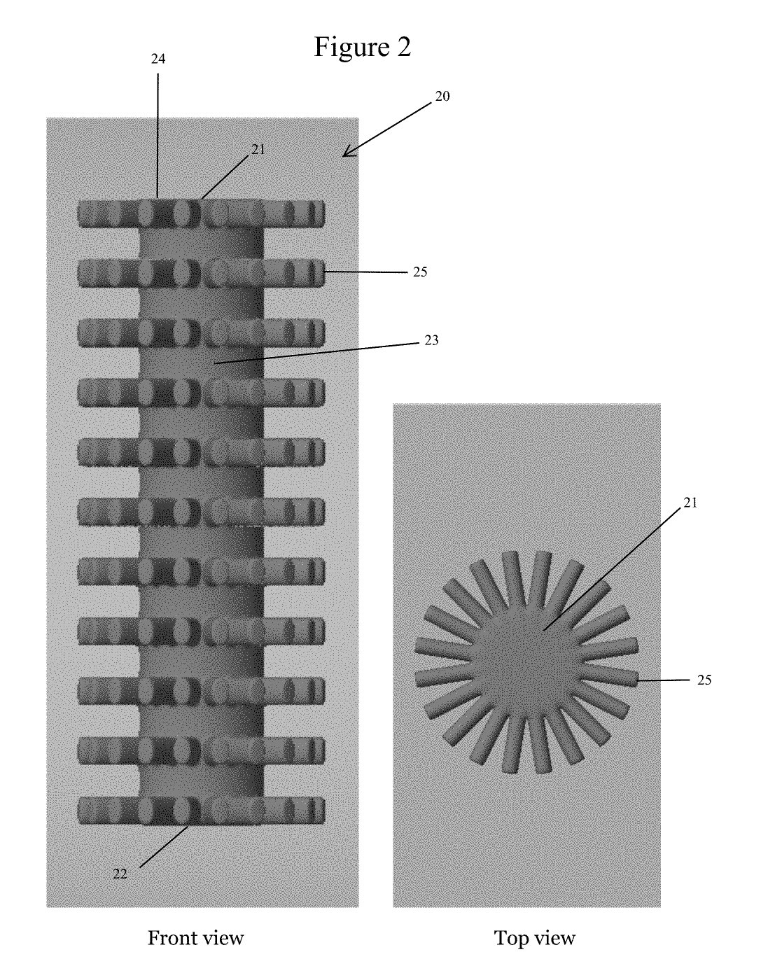

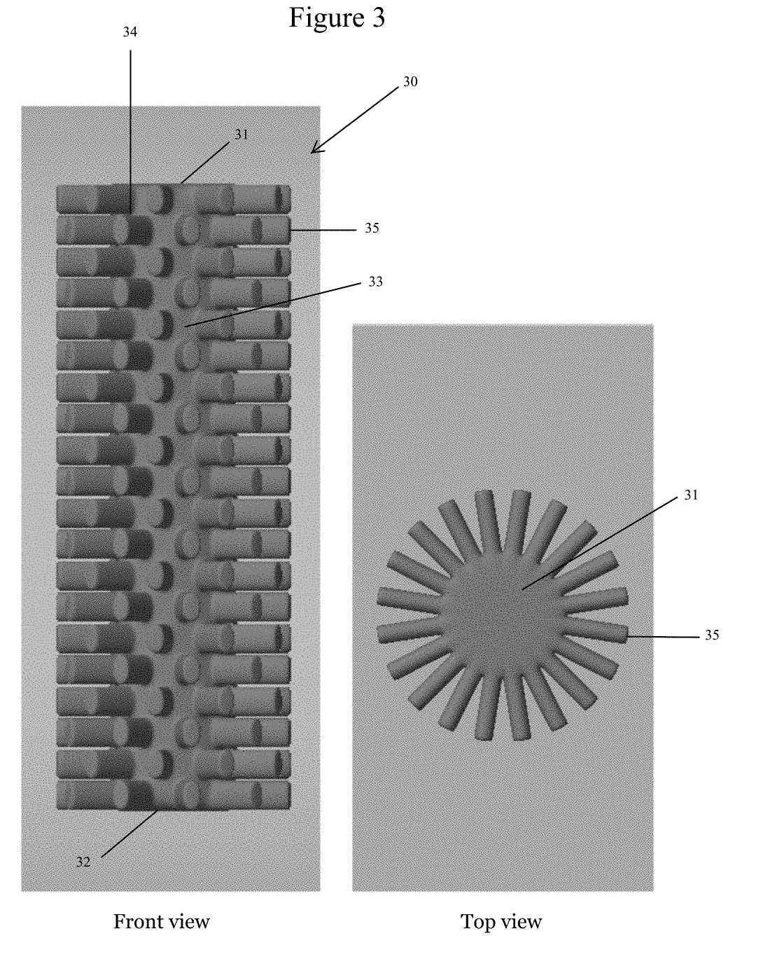

FIGS. 2 and 3 depict alternate implementations of the apparatus of the present disclosure. In FIG. 2, biopsy site marker 20 may comprise a metallic, rod-shaped cylinder having a top surface 21, a bottom surface 22, and a body 23. Body 23 is approximately three to four millimeters in length. The top and bottom surfaces of biopsy site marker 20 are each approximately 1.1 mm in diameter. Spokes 25 extend radially from the body 23 in vertical rows 24. Each row 24 comprises an equally-numbered plurality of spokes 25, and the spokes 25 are equally spaced along each row 24. Moreover, each vertical row 24 is equilaterally and radially spaced around the body 23 of biopsy site marker 20. The plurality of spokes 25 of biopsy site marker 20 results in a textured and reflective surface morphology that may reliably reproduce twinkling artifact when imaged under Doppler ultrasound, thereby improving visibility of the biopsy site marker 20 on ultrasound.

In similar fashion, FIG. 3 depicts biopsy site marker 30. Biopsy site marker 30 may also comprise a metallic, rod-shaped cylinder having a top surface 31, a bottom surface 32, each approximately 1.1 mm in diameter, and a body 33, which is approximately three to four millimeters in length. Spokes 35 extend radially from the body 33 in vertical rows 34. Each row 34 comprises an equally-numbered and equally-spaced plurality of spokes 35. The spokes 35 in adjacent rows are positioned staggered from one another. Moreover, each vertical row 34 is equilaterally and radially spaced around the body 33 of biopsy site marker 30. The plurality of spokes 35 of biopsy site marker 30 results in a textured and reflective surface morphology that may reliably reproduce twinkling artifact when imaged under Doppler ultrasound, thereby improving visibility of the biopsy site marker 30 on ultrasound.

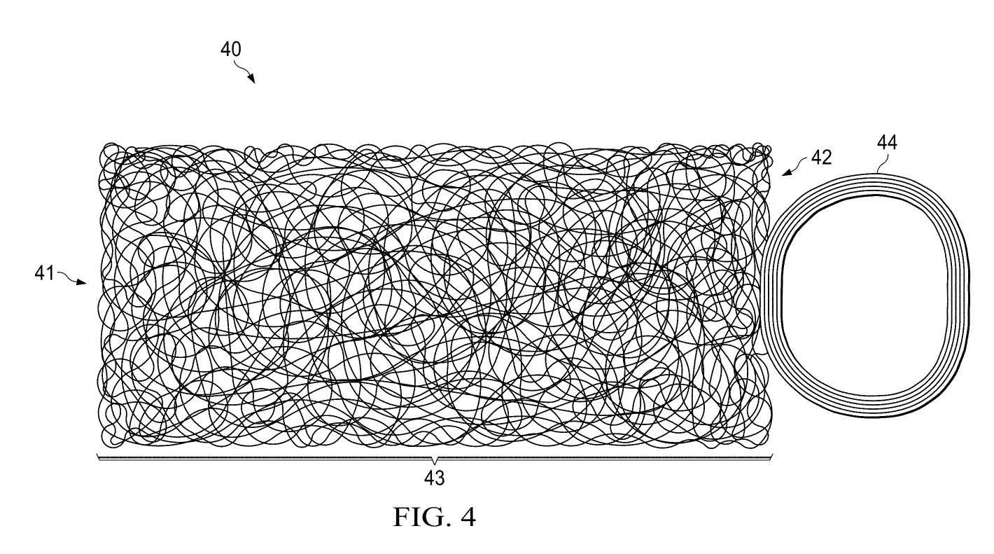

FIG. 4 is a biopsy site marker 40 according to yet another implementation of the present disclosure. Biopsy site marker 40 may comprise a metallic, coiled wire (approximately 0.012 mm diameter wire) with an overall cylindrical body shape. Biopsy site marker 40 may comprise a top portion 41, a bottom portion 42, each approximately 1.1 mm in diameter, and a body 43 which is approximately 3-4 mm in length. Attached to the bottom portion 42 of the artifact-inducing body is an identifying metallic shape 44, which allows a medical practitioner to readily identify the marker on mammographic images. Although the coiled wire of biopsy site marker 40 is depicted as a cylinder and metallic shape 44 is depicted as a loop in FIG. 4, it may be understood by one of ordinary skill in the art that biopsy site marker 40 and metallic shape 44 may comprise any variety of shapes. For example, biopsy site marker 40 may be shaped as a sphere, an ovoid, a cuboid, or other three-dimensional shape. Metallic shape 44 may also comprise any shape, including a triangle, a square, a diamond, heart, or other shape. Because of the textured and reflective surface morphology of the coiled wire, biopsy site marker 40 may reliably reproduce twinkling artifact when imaged under Doppler ultrasound, thereby improving visibility of the biopsy site marker 40 on ultrasound.

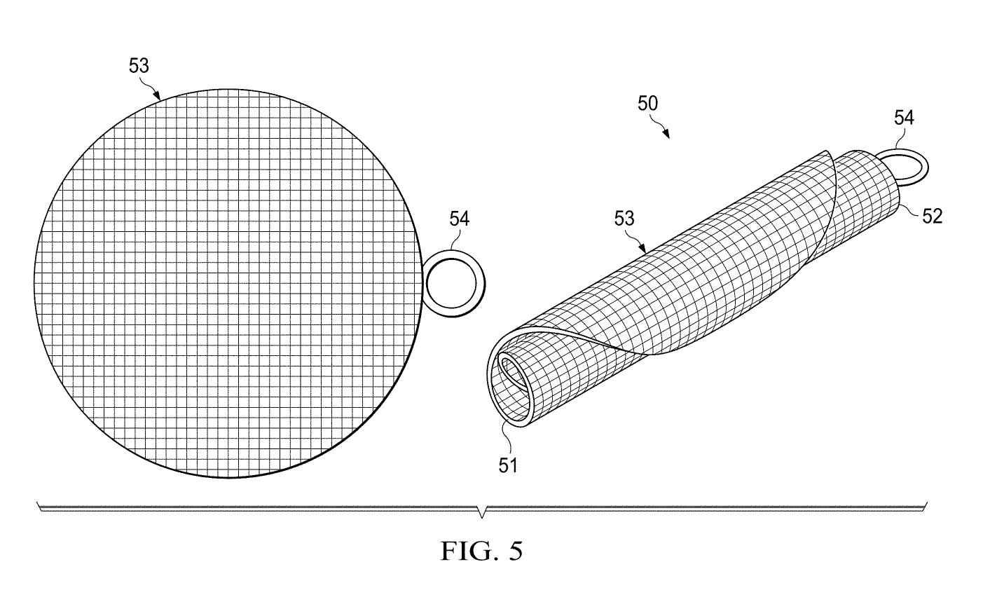

FIG. 5 is a biopsy site marker 50 according to yet another implementation of the present disclosure. Biopsy site marker 50 may comprise a metallic, fine-wire mesh which may be shaped, inter alia, as a flat disk 53. Because of the surface morphology of the wire mesh, biopsy site marker 40 may reliably reproduce twinkling artifact when imaged under Doppler ultrasound. Attached to one end of the wire mesh disk 53 is an identifying metallic shape 54, which allows a medical practitioner to readily identify the marker on mammographic images. Although the wire mesh disk 53 is depicted as a disk and metallic shape 54 is depicted as a loop in FIG. 5, it may be understood by one of ordinary skill in the art that wire mesh disk 53 and the metallic shape 54 may comprise any variety of shape, including a triangle, a square, a diamond, oval, or other shape. In order to deploy wire mesh disc 53 into tissue, disk 53 may be folded or rolled and then inserted into a biopsy marker deployment device. In its folded or rolled state, biopsy site marker 50 may comprise a top portion 51 and a bottom portion 52, each having an approximate diameter of 1.1 mm. The length of the biopsy site marker 50 corresponds to the diameter of the disk 53, which is approximately 3-4 mm. Upon deployment into tissue, the wire mesh disc 53 may unfold into its original shape, or may be configured to remain in its folded or rolled shape.

The textured and reflective surface morphology of biopsy site markers 10, 20, 30, 40, 50 may reliably reproduce twinkling artifact when imaged under Doppler ultrasound, thereby enabling ultrasound detection of biopsy site markers 10, 20, 30, 40, 50 when implanted in tissue. The unique morphology of biopsy site markers 10, 20, 30, 40, 50 eliminates the need to surround the marker with large absorbable material to improve ultrasound visibility. Rather, the ability of markers 10, 20, 30, 40, 50 to produce the twinkling artifact is intrinsic to their engineered structure. Moreover, the ultrasound visibility of markers 10, 20, 30, 40, 50 is not time dependent; the markers will remain readily visible under Dopplar ultrasound throughout their life. Moreover, biopsy site markers 10, 20, 30, 40 may also be visible on mammogram and MRI as they are made of standard titanium and/or stainless steel materials.

With further reference to the biopsy site markers described in conjunction with FIGS. 1-4, a preferred method for detecting a biopsy site marker in human tissue will now be described. First, a biopsy site marker (such as that depicted in FIGS. 1-5 as elements 10, 20, 30, 40, 50) having a textured and reflective surface may be inserted into human tissue proximate to a location where a tissue sample was removed for biopsy. The biopsy site marker may be inserted by any means known or understood by one of ordinary skill in the art. For example, biopsy site marker may be inserted by injection via, e.g., a 17-gauge needle deployment device. In particular, the biopsy site marker may be placed within the hollow needle of the deployment device. The needle deployment device is then inserted into the appropriate tissue location of a patient, and the biopsy site marker may be injected through the needle into the specific tissue location. At any time after insertion of the marker into the tissue, the marker may be imaged and thereby detected using Color Doppler sonography as described herein.

Specifically, Color Doppler sonography uses a transducer to send and receive high-frequency sound waves. In typical applications, the sound waves bounce off solid objects such as blood cells, and any movement of such cells causes a change in pitch of the reflected sound waves. This, in effect, allows a medical practitioner to evaluate and assess blood flow. Within the context of imaging stationary tissue, irregular and reflective objects analyzed under Color Doppler sonography produce a rainbow of colors or a rapid alternation in color—called twinkling artifact—caused by Doppler signal aliasing. Aliasing occurs when flow velocity exceeds the velocity scale set on a sonography machine. Therefore, biopsy site marker 10, 20, 30, 40, 50 having a textured, reflective surface is seen under ultrasound as a twinkling artifact, i.e., a focus of multiple colors simulating flow, even though there is no real flow. Because twinkling artifact may additionally be enhanced by machine parameters, the appearance of twinkling artifact may be maximized by a variety of optimal machine settings or parameters. As such, the biopsy site marker may be readily detected and identified based on the twinkling artifact produced by the marker under Doppler ultrasound.

According to another implementation, the afore-described method of detecting biopsy site markers may also act as a method for site marker localization. Instead of using a wire localization, the biopsy site marker 10, 20, 30, 40, 50 which produces a twinkle artifact can be localized using an ultrasound transducer, i.e., the marker may be visually identified using a Doppler ultrasound probe.

It is to be understood that the implementations are not limited to particular apparatus or methods described which may, of course, vary. For example, the biopsy site markers need not be limited to the specific designs disclosed herein. There are many other variations, iterations, and designs of biopsy site markers that may result in textured and/or irregular surfaces which would allow for the presence of twinkling artifact under Dopplar ultrasound. Additionally, the present disclosure is not limited to imaging by Doppler sonography, but may entail any imaging modality which may produce twinkling artifact. Moreover, the present disclosure is not limited to use with any particular type of tissue or location within the body. Moreover, the disclosure defined by the above paragraphs is not to be limited to particular details set forth in the above description, as many apparent variations thereof are possible without departing from the spirit or scope of the present disclosure. It is also to be understood that the terminology used herein is for the purpose of describing particular implementations only and is not intended to be limiting.

Although the present disclosure has been set forth in detail, it should be understood that various changes, substitutions, and alterations may be made herein without departing from the spirit and scope of the disclosure as defined by the appended claims. Moreover, the scope of the present application is not intended to be limited to the particular embodiments of the process, machine, manufacture, composition of matter, means, methods, and steps described in the specification. As one of ordinary skill in the art will readily appreciate from the disclosure, the processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method for detecting a biopsy site marker in tissue comprising:
inserting a biopsy site marker having a textured surface into the tissue;
imaging the tissue having the marker using Doppler sonography; and
identifying the marker based on a twinkling artifact appearing on the Doppler sonography.

2. The method of claim 1, wherein the biopsy site marker is reflective.

3. The method of claim 1, wherein the textured surface comprises:
a plurality of spokes.

4. The method of claim 1, wherein the textured surface comprises:
at least one coiled wire.

5. The method of claim 1, wherein the textured surface comprises:
a wire mesh.

6. An apparatus comprising:
a biopsy site marker for placement into tissue, the marker having at least one textured surface capable of creating twinkling artifact when imaged using Doppler sonography.

7. The apparatus of claim 6, wherein the biopsy site marker is reflective.

8. The apparatus of claim 6, wherein the textured surface comprises a plurality of spokes.

9. The apparatus of claim 6, wherein the textured surface comprises at least one coiled wire.

10. The apparatus of claim 9, wherein the at least one coiled wire is formed into a three-dimensional shape comprising:
a top portion;
a bottom portion; and
a body.

11. The apparatus of claim 10, wherein the coiled wire formed into a three-dimensional shape further comprises:
a metallic shape coupled to the bottom portion.

12. The apparatus of claim 6, wherein the textured surface comprises a wire mesh.

13. The apparatus of claim 12, wherein the wire mesh is disk-shaped.

14. The apparatus of claim 12, wherein the wire mesh is formed into a three-dimensional shape comprising:
a top portion;
a bottom portion; and
a body.

15. The apparatus of claim 14, wherein the wire mesh further comprises:
a metallic shape coupled to the bottom portion.

16. A method for detecting a biopsy site marker comprising:
implanting a biopsy site marker having a textured surface into tissue;
imaging the tissue having the biopsy site marker using Doppler sonography; and
identifying the biopsy site marker based on a twinkling artifact appearing on the Doppler sonography, the twinkling artifact resulting from Doppler signal aliasing produced by the textured surface.

17. The method of claim 16, wherein the biopsy site marker is reflective.

18. The method of claim 16, wherein the textured surface comprises:
a plurality of spokes.

19. The method of claim 16, wherein the textured surface comprises:
at least one coiled wire.

20. The method of claim 16, wherein the textured surface comprises:
a wire mesh.
